(19) United States
(12) Plant Patent Application Publication
(10) Pub. No.: US 2025/0204293 P1
(43) Pub. Date: Jun. 19, 2025

(54) Rubus plant named 'BT RASseven'

(50) ;

(71) Applicant: Diego Ioriatti, Baselga di Pine (IT)

(72) Inventor: Diego Ioriatti, Baselga di Pine (IT)

(21) Appl. No.: 18/964,279

(22) Filed: Nov. 29, 2024

(30) Foreign Application Priority Data
Dec. 13, 2023 (QZ) 2023/2724

(51) Int. Cl. A01H 6/74; A01H 5/00; A01H 5/08

(52) U.S. Cl. CPC A01H 6/7499; A01H 5/00; A01H 5/08
USPC PLT/204

(57) ABSTRACT

A new and distinct cultivar of Rubus plant named ‘BT RASseven’ is disclosed, characterized by high quality fruits in terms of taste, aroma, sugar level and shelf life. Plants are semi-erect, including the lateral branches. Fruit has a clear light red color which is maintained during post-harvest. Plants have dense pubescence at the base of the plant. The new cultivar is a Rubus, intended for commercial fruit production.

Latin name of the genus and species: Rubus idaeus.

Cultivar denomination: ‘BT RASseven’.

### BACKGROUND OF THE INVENTION

The new Rubus cultivar is a product of a planned breeding program conducted by the inventor in Baselgia di Pine, Italy. The objective of the breeding program was to produce new Rubus idaeus varieties for commercial applications. The cross resulting in this new cultivar was made during September of 2019.

The seed parent is a proprietary, unpatented cultivar referred to as Rubus ‘BT17AC190’. The pollen parent is the unpatented proprietary cultivar Rubus ‘BPDRE’. The new cultivar was selected during the August of 2021 by the inventor in a group of seedlings resulting from the 2019 crossing, in a research greenhouse in Baselgia di Pine, Italy.

The inventor has filed an application with the CPVO Dec. 13, 2023, having application number 2023/2724. This disclosure falls within the exception allowed under 102(b)(1).

Asexual reproduction of the new cultivar was performed by vegetative suckers. This was first performed at a research greenhouse in Baselgia di Pine, Italy during February of 2022 and has shown that the unique features of this cultivar are stable and reproduced true to type in multiple successive generations.

### SUMMARY OF THE INVENTION

The cultivar ‘BT RASseven’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘BT RASseven’. These characteristics in combination distinguish ‘BT RASseven’ as a new and distinct Rubus cultivar:
1. High quality of the fruits in terms of taste, aroma, sugar level and shelf life.
2. Clear color of the fruits.
3. Semi-upright architecture of the canes and lateral branches.
4. Good tolerance to most raspberry pests and diseases.
5. Dense pubescence at the base of the plant. .

### PARENT COMPARISON

Plants of the new cultivar ‘BT RASseven’ are similar to plants of the seed parent, in most horticultural characteristics, however, plants of the new cultivar ‘BT RASseven’ differ in the following:
1. The new cultivar produces fruit which are more homogenous in size.
2. The new cultivar has a lighter fruit color.
3. The new cultivar has less spinescence.
4. The new cultivar produces shorter fruit laterals and a more erect architecture.
5. Budbreak occurs on the floricane of the new cultivar more regularly than the seed parent cultivar.

Plants of the new cultivar ‘BT RASseven’ are similar to plants of the pollen parent, in most horticultural characteristics, however, plants of the new cultivar ‘BT RASseven’ differ in the following:
1. The new cultivar produces taller primocane.
2. The new cultivar has lower remontancy.
3. The new cultivar fruits later on primocane.
4. The new cultivar produces less branches than the pollen parent cultivar.
5. The new cultivar produces fruit which is more conical in shape than fruit of the pollen parent.

### COMMERCIAL COMPARISON

Plants of the new cultivar ‘BT RASseven’ are comparable to the cultivar Rubus ‘BT RASTWO’, U.S. Plant Pat. No. 33,447. The two Rubus varieties are similar in most horticultural characteristics, however, the new cultivar ‘BT RASseven’ differs in the following:
1. The new cultivar produces more branches.
2. The new cultivar has a lower chill requirement.
3. The new cultivar has a later primocane fruit harvest.
4. The new cultivar produces firmer fruit with a better shelf life.
5. The new cultivar has thinner leaves.

Plants of the new cultivar ‘BT RASseven’ can also be compared to the commercial cultivar Rubus ‘BP1’ U.S. Plant Pat. No. 22,459. These varieties are similar in most horticultural characteristics however, the new cultivar ‘BT RASseven’ differs in the following:
1. The new cultivar is more productive both on primocane and on floricane harvest.
2. The new cultivar produces less spinescence.
3. The new cultivar has a more upright architecture.
4. The new cultivar produces fruit with a lighter and more stable color in postharvest.
5. The new cultivar produces less primocanes from the ground.

### BRIEF DESCRIPTION OF THE PHOTOGRAPHS

The photographs were taken using conventional techniques and although colors May appear different from actual colors due to light reflectance it is as accurate as possible by conventional photographic techniques. Colors in the photographs may appear to differ slightly from the color values cited in the botanical description, which accurately describe the colors.

FIG. 1 illustrates in full color harvested fruit of ‘BT RASseven’.

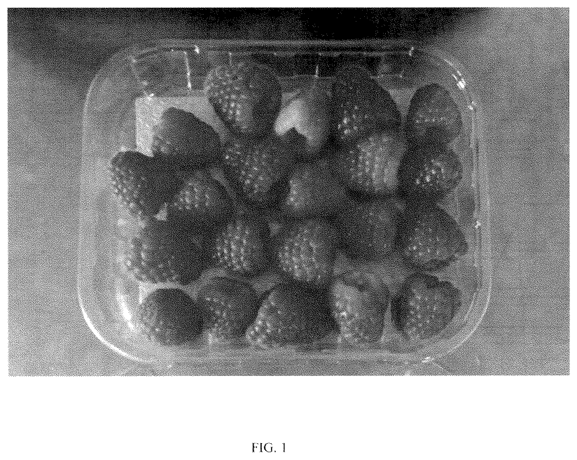

FIG. 2 illustrates in full color the base of a plant of ‘BT RASseven’, with typically dense pubescence.

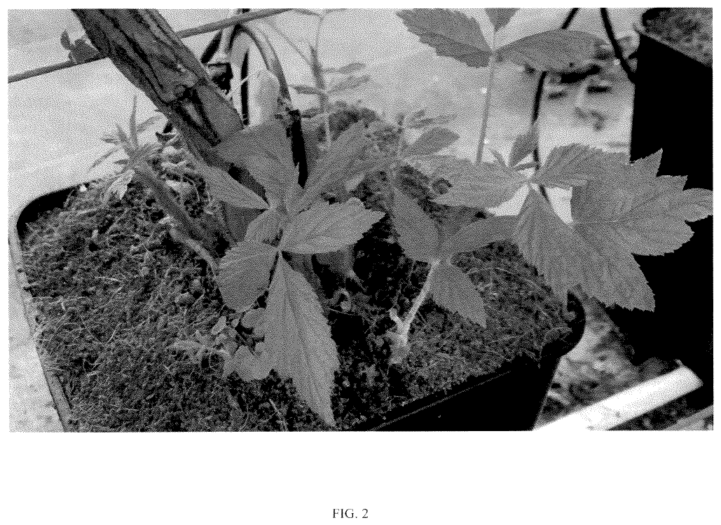

FIG. 3 illustrates in full color typical plants of ‘BT RASseven’. Age of the plants is approximately 1 year old grown in a poly hoop house.

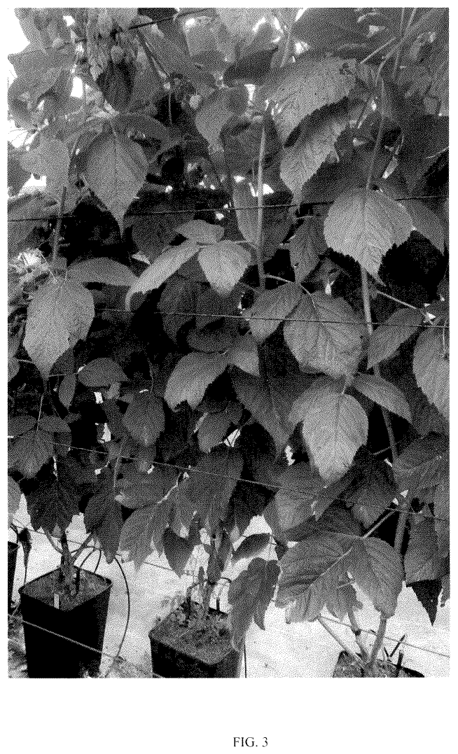

### DETAILED BOTANICAL DESCRIPTION

In the following description, color references are made to the Royal Horticultural Society Colour Chart 2007 except where general terms of ordinary dictionary significance are used. The following observations and measurements describe ‘BT RASseven’ plants which were grown in Borgo Valsugana, Trento, Italy. Plants were grown in a season tunnel, given approximately 30% shading during Summer. The growing temperature ranged from about 15° C. to 34° C. during the day and from about 8° C. to 22° C. during the night. Measurements and numerical values represent averages of typical plant types. Plants are approximately 1 year old.
Botanical classification: Rubus idaeus ‘BT RASseven’.
Propagation
   Time to Initiate Roots.—About 35 days at approximately 16° C.
   Time to Produce a Rooted Cutting.—About 45 days at 18° C.
Plant
   Age of plant described.—About 1 year.
   Plant Spread.—Approximately 70 cm.
   Plant Height.—Approximately 200 cm.
   Habit.—Semi-upright.
   Primocane length.—Approx. 230 cm.
   Primocane color.—Near RHS Yellow-Green N151B.
   Anthocyanin coloration present.—Not typical.
   Dormant cane color.—Near RHS Greyed-Orange 164A.
   Primocane diameter.—Average 1.2 cm.
   Internode length.—Average 5 cm.
   Spines.—Approximately density/quantity of spines: Extremely high density of small spines at the base level. About 30 to 40 cm of height, moderate density of spines, approximately 3 to 4 per cm of stem length at 1 meter of height. Length: Average range 2 to 3 mm. Color: Near RHS Brown 200A. Shape: Short deltate.
Foliage:
   Leaf.—Arrangement: Compound opposite. Quantity: 9-10 per stem. Average Length: Approximately 26 cm. Average Width: Approximately 18 to 20 cm.
   Leaflet.—Quantity: Most common quantity of leaflets per leaf: 3. Average Length: Approximately 14-19 cm. Average Width: Approximately 7-10 cm. Shape of blade: Overall broad deltate. Apex: Acuminate. Base: Broad rounded. Margin: Double serrate. Texture of top surface: Coriaceous. Texture of bottom surface: Coriaceous. Pubescence: Low. Leaf internode length: 6-9 cm. Color: Young foliage upper side: Near RHS Yellow-Green 144B. Young foliage under side: Near RHS Green 142B. Mature foliage upper side: Near RHS Green 141B. Mature foliage under side: Near RHS Green 134B. Venation: Type: Cross veniculate, pinnate along main axis. Venation color upper side: Near Yellow-Green 144C. Venation color under side: Near Yellow-Green 144D. Petiole: Length: 6 to 8 cm. Width: 3 mm. Color: Near RHS Yellow-Green N144C. Texture: Glabrous. Rachis: Length: 12-13 cm. Width: 3-4 mm. Color: Near RHS Yellow-Green N144C. Texture: Glabrous. Stipules: Not observed.
Flower:
   Floral bud burst date.—Approximately July 25 for primocane flowers.
   Flowering season.—Begins around August 20 in Verona, Italy.
   Inflorescence.—Flowers per: 15-24.
   Flower size.—Diameter: Approximately 2 cm. Length: Approximately 1 cm.
   Peduncle.—Length: 5 cm. Diameter: 0.2 cm. Color: Near RHS Yellow-Green N144C. Texture: Glabrous.
   Pedicel.—Length: 1.3 cm. Diameter: 2 mm. Color: Near RHS Yellow-Green N144C. Texture: Glabrous.
   Petals.—Petal arrangement: Rotate. Length: 9 mm. Width: 5 mm. Shape: Semi-cuneate. Margin: Entire. Apex: Rounded. Base: Rounded. Petal quantity: 5. Texture: Glabrous.
   Color: Petals: When opening.—Upper surface: Near RHS White N155B. Lower surface: Near RHS White N155B. Fully opened: Upper surface: Lower surface: Near RHS White N155B. Near RHS White N155B.
   Bud.—Shape: Ovate. Length: Approximately 6 mm. Diameter: Approximately 4 mm. Color: Near RHS Greyed-Orange 166B. Sepals: Length: 1.5 cm. Width: 6 mm. Shape: Narrow deltate with apiculate apex. Color. Interior Surface: Near RHS Green 140C. Exterior Surface: Near RHS Green 141B. Texture: Glabrous.
Reproductive organs:
   Androecium.—Stamens: Number: 120 to 135. Filament length: Approximately 6 mm. Filament color: Near RHS Green-White 157A.
   Anthers.—Length: 0.8 mm. Shape: Ovate. Color: Near RHS Greyed-Orange 164A. Pollen: Quantity: Moderate. Color: Near RHS White N155C.
   Pistil.—Number: 135 to 145. Length: Approximately 4 mm. Style: Length: Approximately 4 mm. Color: Near RHS Green-White 157A.
Fruit:
   Market Use.—Fresh consumption.
   Length.—4.5 to 5 cm.
   Diameter.—2.7 to 3 cm.
   General shape in lateral view.—Round-conical.
   Color.—Near RHS Red 43B, 43C and 43D.
   Average Druplets per berry.—180.
   Average weight per berry.—5.5 to 6 grams.
   Fruit Bearing on Floricane/Primocane or both.—Both.
   Fruit begins ripening.—Primocane about August 25th; floricane about April 15th.
   Fruiting season.—Primocane September 15 to November; floricane June to mid-July.
   Yield per hectare.—15 to 19 ton/ha in primocane; 23 to 24 ton/ha in floricane.
   Other Fruit characteristics.—Clear colored fruit of medium size with very slow darkening compared to most commercial varieties. Very stable clear color also maintained after harvest.
Other characteristics:
   Disease/pest resistance.—Observed good tolerance for raspberry aphid, Amphorophora idaei and leafhoppers, Edwardsiana rosae.
   Temperature Tolerance.—Maximum 36° C. during pollination and harvest. Good tolerance to winter frosts to −15° C.

What is claimed is:

1. A new and distinct cultivar of Rubus plant named ‘BT RASseven’ as herein illustrated and described.
